(19) United States
(12) Patent Application Publication
(10) Pub. No.: US 2012/0296365 A1
(43) Pub. Date: Nov. 22, 2012

(54) Method and Apparatus for Buoyant Gastric Implant

(75) Inventor: Khoi Minh Nguyen, Tustin, CA (US)

(73) Assignee: Endobese, Inc.

(21) Appl. No.: 13/474,585

(22) Filed: May 17, 2012

Related U.S. Application Data
(60) Provisional application No. 61/487,184, filed on May 17, 2011.

(51) Int. Cl. A61F 2/04

(52) U.S. Cl. 606/192

(57) ABSTRACT

A buoyant, expandable intragastric device is provided that can be inserted into the stomach of a patient. The device is inflated, or expanded, with gas or other low density material to partially fill the stomach and enabling the device, or implant, to be buoyant within the stomach by floating toward the highest location possible relative to the contents of the stomach and the configuration of the stomach walls. The implant moves around as the body changes orientation or as the stomach contents change. Therefore, continual impingement on the same tissues of the gastrointestinal tract is minimized. The implant, being buoyant and floating to the top of the stomach, can beneficially generate increased pressure on, or stretching of, the tissues at the top of the stomach and the vagal nerves causing signals to the brain indicating that the stomach is full.

### RELATED APPLICATIONS

This application claims priority to copending U.S. Provisional Patent Application 61/487,184 filed May 17, 2011.

### FIELD OF THE INVENTIONS

The present invention relates to medical devices and procedures and more particularly to expandable, buoyant intragastric devices for insertion, positioning, and deployment into a patient's body cavity, such as the stomach, intestine or gastrointestinal track, as well as removal therefrom, for filling space to provide the patient with a feeling of satiety or fullness.

### BACKGROUND OF THE INVENTIONS

Obesity is a chronic, multi-factorial disease that develops from an integration of genetic, environmental, social, behavioral, physiological, metabolic, neuron-endocrine and psychological elements. This disease is related to such conditions as GERD, high blood pressure, elevated cholesterol, diabetes, sleep apnea, mobility and orthopedic deterioration, and other consequences, including those limiting social and self image and those affecting the ability to perform certain everyday tasks. Traditional weight loss techniques, such as diet, drugs, exercise, etc., are ineffective with many of these patients. The only viable alternative for many patients is surgical intervention.

### SUMMARY

The devices and methods described below provide for the treatment of obesity. The buoyant intragastric device is designed for simple deployment and removal into the stomach for the treatment of obesity. The intragastric device includes an expandable member and at least one self-sealed port for inflation of the device, such as flapper valve, a spring valve or any other suitable valve that provides inflation access through the wall of the device.

A buoyant, expandable intragastric device is provided that can be inserted into the stomach of a patient. The device can be deployed and/or removed through trans-esophageal approaches. The device is inserted and then inflated, or expanded, with gas or other low density material to partially fill the stomach and enabling the device, or implant, to be buoyant within the stomach by floating toward the highest location possible relative to the contents of the stomach and the configuration of the stomach walls. The implant moves around as the body changes orientation or as the stomach contents change. Therefore, continual impingement on the same tissues of the gastrointestinal tract is minimized. The implant, being buoyant and floating to the top of the stomach, can beneficially generate increased pressure on, or stretching of, the tissues at the top of the stomach and the vagal nerves, thereby causing signals to the brain indicating that the stomach is full. These early signals of stomach fullness, coupled with reduced food intake, provide the recipient with the tools necessary to prevent excessive caloric intake.

The devices and methods described below provide greater effectiveness, less invasiveness, reversibility, and other needs by providing for new and improved methods and apparatus for implantation and removal of devices into the gastrointestinal system of a mammalian patient. The disclosed system further provides methods and devices for implantation in the stomach of a patient that can be deployed in a minimally invasive manner through clinically established techniques, such as the technique used during a percutaneous endoscopic gastrostomy (PEG) tube placement, a procedure that includes trans-esophageal endoscopy.

The devices and methods described below provide greater access to procedures and devices by patients who might not otherwise be treated surgically as severely or morbidly obese, such as with a BMI of greater than 35 kg/m³, but who may just be moderately obese or overweight with a BMI of between 25 to 35 kg/m³. In addition, patients who require more invasive surgery for an unrelated ailment may need a minimally or non-invasive way to lose the weight prior to their more invasive procedure, thereby reducing the risks associated with general anesthesia, or otherwise enabling the more invasive procedure.

In some configurations, a buoyant, expandable intragastric device is provided that can be inserted into the stomach of a patient. The device is inflated, or expanded, with gas or other material which is less dense than water. Thus, the device, or implant, is buoyant in water and its position is maintained within the stomach by floating toward the highest location possible relative to the stomach contents and the configuration of the stomach walls, which expand and contract. The buoyant implant can move around as the body changes orientation or as the apparent stomach size changes as a result of content change, etc. Therefore, continual impingement on the same tissues of the gastrointestinal tract is minimized. Certain vagus (or vagal) nerves are located in the gastroesophogeal region at the top of the stomach where the esophagus joins the stomach. These vagal nerves sense stretching of tissues at the top of the stomach, due to the presence of stomach contents in that area, and these signals indicate to the brain that the stomach is full, thus providing a sense of satiety. The implant, being buoyant and floating on the stomach contents to the top of the stomach, can beneficially generate increased pressure on, or stretching of the tissues embedded with vagal nerves, causing the stomach to send signals to the brain that the stomach is full. These early signals of stomach fullness, following reduced food intake, provide the recipient with the tools necessary to prevent excessive caloric intake.

In some configurations, the implant can serve to divide the stomach into two virtual parts, the upper part and the lower part, with the implant positioned between. Any food entering the stomach in solid form resides above the implant and generates early sensations of fullness on the vagus nerves near the top of the stomach. As the food is digested and becomes liquid, it passes through channels in the perimeter or the center of the implant to reach the lower part of the stomach and continue through the digestive tract.

In another configuration, the apparatus described below provides an expandable intragastric device that consists of multiple layers of materials. The inner, or barrier, layers are configured for structural integrity as well as being a gas barrier. The outer layers are configured to provide minimal tissue abrasion to minimize negative interaction with the internal surface of the gastrointestinal tract and its individual organs as well as resisting the erosive contents of the gastrointestinal tract. The gas barrier layer or layers can be selected to perform as either unidirectional or bi-directional. In the unidirectional configuration, gas can enter the buoyant implant but it cannot migrate out through the layers of the implant to the exterior. In the bi-directional configuration, gas can enter into, as well as permeate out of, the buoyant, expandable implant.

In yet another configuration, the apparatus described below provides for a buoyant, expandable intragastric device that maintains its expanded shape and desired volume, independent of any small leaks that may develop over time, without the requirements of refilling. Furthermore, in the event of leaks, the implant prevents against migration. The materials used to fill the implantable device are chosen such that if a leak occurs, the leaked filler material, gas, gel, gas generator material or compound, gas enhancer, filaments, or other substance, does not contaminate the patient with toxic materials or cause any blockage of the gastrointestinal tract.

The buoyant, expandable implants as described below are configured to simplify installation and to facilitate removal. Filler valves and the seats to which they are secured engage the various configurations of expandable implants such that when the implant is fully expanded, the valve seat flange is nearly flush with the edge of the implant exterior surface. When the implant is deflated prior to removal, the valve seat flange separates from the limp implant exterior surface, providing a point of engagement for any suitable surgical tool such as a standard surgical snare to engage the valve seat flange and remove the implant.

The apparatus described below also provides for methods and apparatus for maintaining a constant volume of the device while it is maintained in the deployed condition or state.

The gastric implant device is a part of a system designed for treating obesity. The buoyant implant may be called a gastric device, implant or balloon and is delivered to the patient's stomach via any suitable endoscopic procedure.

The buoyant, expandable implant, in its first, deflated or collapsed state, is attached to the delivery catheter as a complete-packaged assembly. Following sedation of the patient, the gastrointestinal (GI) physician, or surgeon, inserts the device into patient's stomach through his or her mouth and esophagus. After the device is positioned (located) at the desired location, the device can be inflated to a second, fully, or partially, inflated configuration. Inflation need not be full or complete and, in some preferred configurations, inflation is partial. This methodology eliminates any underfill or overfill situation which could cause the device to become improperly positioned within the stomach. The disclosed apparatus does not require volumetric filling but rather functions with partial filling to within certain, controlled volume and or pressure ranges.

The device is then detached from the delivery catheter assembly by an action performed by the operator at the proximal end of the delivery catheter; and the delivery catheter assembly is removed from patient leaving the inflated gastric device inside the stomach. The gastric implant serves as a buoyant space occupier to help the patient feel a sensation of fullness thus reducing the sense of hunger leading to less food intake.

The implant, being buoyant and floating on the contents of the stomach, pushes against the vagal nerves near the top of the stomach, stretching those tissues and causing early sensations of fullness for the recipient. Yet another benefit is that the stomach may be divided into two parts by the implant, causing solid food to preferentially collect above the implant, forcing the solid food to stretch the vagal nerves in the gastroesophogeal region of the stomach. This stretching of the top of the stomach causes a feeling of fullness or satiety much earlier than if the device was not present or served merely as a space-filling mechanism. The food temporarily trapped or collected above the implant in the upper stomach compartment eventually migrates past or through the implant to the lower compartment where it continues to move through the digestive tract.

In some configurations, an inflatable or otherwise expandable space occupying device is provided that can be delivered through the patient's mouth in a trans-esophageal procedure and deployed within the patient's stomach or other gastrointestinal tract region. The device comprises an expandable member with at least one gas-generator component. The gas-generator can be in the form of liquid or solid state material, or material combining both liquid and solid state. The gas-generator as discussed herein may also be understood as a gas enhancer.

A suitable gas-generator, or catalyst may be Perfluoropentane, Perfluorohexane, or the like, in their liquid state. These materials are specified to evaporate and to stop or cease evaporating, thus producing gas within specific vapor pressure ranges and temperatures to maintain the shape and internal pressure of implanted device without exceeding the pressure limits of the implanted device. By maintaining constant vapor pressure, the interior of the implant retains a controlled internal pressure and, thus, maintains constant volume, even if fluid leakage occurs. Examples of the target pressure range can be from about 0 to about 20 PSI and more specifically from about 0 to about 5 PSI. The temperature range will generally remain about body temperature or about 35 to about 39 degrees centigrade, although some natural fluctuation around this temperature occurs within the stomach. These materials are also selected because their large molecular size relative to air or other materials which permits use of a relatively porous implant skin that would permit escape of smaller molecules.

In other configurations, the gas-generator or catalyst comprises other materials to extract gases from the outside environment; in this case the contents of the stomach, to fill the implanted device up-to specified volume, pressure, or shape.

In other configurations, the expandable device is pre-filled with hydrogel material. The hydrogel material is hydrophilic and swells, or increases in volume in response to water uptake from the environment. The hydrogel can be in the form of laminates of material on the inside of the implant, a solid mass of material, or a plurality of small beads, pellets, filaments, or balls of either solid or hollow construction.

In other configurations, the interior volume of the implant comprises filler fabricated from at least two different materials. Some or all of these materials can be present within the interior volume prior to implant or injected into the implant following placement within the patient. In a preferred configuration, the second part, or final part of a multiple part system, is injected into the interior volume of the implant following placement within the stomach. A chemical reaction occurs between the materials within the interior volume of the implant generating the gas necessary to fill the interior volume of the implant. In other configurations, the chemical reaction creates a foam matrix or a plurality of bubbles that fill and expand the interior volume. In these configurations, injection of yet another chemical or material into the implant's interior volume can cause a reaction to bind the gas into a liquid and deflate the implant in preparation for removal.

The expandable member can be constructed of a composite structure or comprise multiple layers of material to achieve desirable surface characteristics and is preferably visible under X-ray visualization. The implant comprises materials that are not heated or moved in the presence of a large magnetic field such as is found with magnetic resonance imaging (MRI).

In addition, the device as described below can have surface features, such as one or more flanges, beads, loops, projections, detents, or tabs to facilitate manipulation, deflation, or removal of the device by grasping instruments or other removal devices.

In other configurations, systems and methods are provided for keeping the balloon's volume, pressure, or both approximately, or substantially, constant with a gas filled device. In some configurations, the implant is filled, partially or completely, with gas created by the gas-generator.

The gas generator can generate between about 0% and 100% of the gas pressure within the interior volume of the implant. The gas generator can generate gas that adds to, or compliments, the pressure of gas, hydrogel, or fluid, already present within the internal volume of the implant. The gas generator can generate gas comprising between about 1% and 20% of the internal pressure of the implant. The gas generator can generate gas pressure comprising between about 10% and 40% of the gas pressure within the internal volume of the implant. The gas generator can generate gas pressure comprising between about 30% and 60% of the gas pressure within the internal volume of the implant. The gas generator can generate gas pressure comprising between about 50% and 100% of the gas pressure within the internal volume of the implant. The percentage of the internal pressure of the implant comprised by the gas created by the gas generator can vary substantially over time to beneficially allow the implant to breath or contract and expand multiple times over the period of implantation within the stomach.

In some configurations of the intragastric devices, the devices separate the stomach into different areas or compartments thus providing a technique for restricting flow of food into a patient's digestive system. In these configurations of the intragastric devices, provisions are made to allow the food trapped in an upper area of the stomach to slowly migrate past the exterior, or through one or more internal channels, of the implant to the lower part of the stomach where the food continues digestion in the balance of the gastrointestinal tract.

In some configurations, systems and methods are provided to maintain the intragastric device, balloon or implant inflated over a long period of time even with some loss or diffusion of inflationary media within the balloon or implant.

In some configurations, systems and methods are provided for dynamic implant performance. A dynamic balloon or implant is dynamically changing its size by the intake and exhaust of gas, apparently breathing. This breathing entails deflating due to gas loss through the implant wall to reduce volume versus vaporizing to increase volume, so stomach contents or other material cannot build-up in the upper areas of the stomach or on the outside of the balloon.

In some configurations, systems and methods are provided for increased patient safety by ensuring the implant does not migrate through the duodenum into the bowel causing obstruction and potentially lethal consequences by floating upward or maintaining buoyancy in the stomach. In some configurations, systems and methods are provided to prevent the implant from obstructing the duodenum or exit to the stomach, thus providing improved safety that gastrointestinal blockage cannot occur as a result of the device implantation.

For purposes of summarizing the invention, certain aspects, advantages, and novel features of the invention have been described herein. It is to be understood that not necessarily all such advantages may be achieved in accordance with any particular configuration of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 is a view of a patient with an implanted buoyant gastric device and an illustration of the installation and removal apparatus.

FIG. 2 is an exploded oblique view of a gastric implant comprising a plurality of layers of thin film and a pre-cut opening hole.

FIG. 3 is an exploded oblique view of the upper and lower segments of an implant comprising an integral valve port.

FIG. 4 is an oblique cross-sectional view of the assembled segments of the gastric implant of FIG. 3 with the valve port inverted.

FIG. 5A is an oblique view of the assembled gastric implant device of FIG. 2.

FIG. 5B is an oblique view of the gastric implant of FIG. 5A following inversion to dispose seams or bonds on the inside of the device along with the valve port.

FIG. 5C is an oblique view of the inverted, gastric implant of FIG. 5B, with the valve port installed in the pre-cut opening hole.

FIG. 5D is an oblique view of the gastric implant of FIG. 5C with a valve installed in the valve port and a coating of hydrophilic hydrogel over at least a portion of the exterior surface of the device.

FIG. 6 is an oblique view of a gastric implant comprising an oval body with an opening at one end of the body and a valve port.

FIG. 7A is an oblique view of a gastric implant comprising an oval body, an opening at one end, a plurality of reinforcing ribs and a valve port.

FIG. 7B is an oblique cross-sectional view of the implant of FIG. 7A further comprising a valve port affixed to the device body in communication with the opening.

FIG. 7C is an oblique view of the implant of FIG. 7A further comprising a hydrophilic coating over at least a portion of the exterior of the implant as well as a valve assembly affixed to the valve port.

FIG. 8A illustrates an oblique view of a gastric implant comprising a stretch blow molded or extrusion blow molded body and ports at each end.

FIG. 8B is an oblique cross-sectional view of the implant of FIG. 8A showing the device with the ports having been trimmed in length and folded or inverted inside the body.

FIG. 8C is an oblique cross-sectional view of the implant of FIGS. 8A and 8B further comprising a valve affixed to each of the ports.

FIG. 9 is an oblique cross-sectional view of a gastric implant comprising a plurality of cells and a centrally located side port.

FIG. 10 is an oblique cross-sectional view side view of a gastric implant fabricated from two molded pouches or bulbs following which the pouches are affixed together in the central region which comprises a valve.

FIG. 11 is an oblique cross-sectional view of a gastric implant constructed from four separately formed segments, which are welded together following which the two halves of the device are inverted, welded together with a flapper valve.

FIG. 12 is an oblique view of the gastric implant of FIG. 11 except with a spring-loaded valve.

FIG. 13A is an oblique view of a molded gastric implant comprising a plurality of cells having one or more valve ports aligned with the major axis of the implant.

FIG. 13B is an oblique view of the gastric implant of FIG. 13A with the valve ports inverted for installation of the valve assemblies.

FIG. 14A is an oblique, exploded cross-sectional view of a buoyant gastric implant having a central valving system operably connected to two valve ports located one at each end of the implant.

FIG. 14B is an oblique cross-sectional view of the buoyant gastric implant of FIG. 14A with ballast weights installed.

FIG. 15 is an oblique cross-sectional view of an intragastric implant with its interior cavity filled, at least in part, with small space-filling structures such as balloons.

FIG. 16A illustrates an gastric implant in a first, narrow, elliptical configuration for placement.

FIG. 16B illustrates a cross-section of the gastric implant of FIG. 16A in a second, larger round configuration.

FIG. 16C illustrates a hydrogel bead or pellet suitable for filling the interior volume of a gastric implant, wherein the bead is in its first, dry, small diameter configuration.

FIG. 16D illustrates the bead or pellet of FIG. 16C in its expanded configuration.

FIG. 16E illustrates the implant of FIG. 16A filled with the pellets of FIG. 16B, before expansion.

FIG. 16F illustrates the implant of FIG. 16B filled with the expanded pellets of FIG. 16D.

FIG. 17 illustrates a gastric implant deployed in the stomach.

FIG. 18A illustrates a gastric implant comprising an annular construction and generally cylindrical outer walls with a central orifice through which food can migrate following digestion by the stomach.

FIG. 18B illustrates a gastric implant comprising annular construction with a central orifice and a more rounded exterior configuration in the direction of the longitudinal axis.

FIG. 19A is an oblique view of a buoyant gastric implant with a removable fill tube sutured to the valve flange.

FIG. 19B is a close-up oblique view of the details of the fill tube, valve and suture attachment of FIG. 19A.

FIG. 20A is an oblique view of a fully inflated, buoyant gastric implant.

FIG. 20B is a close-up cross-section view of the valve assembly, valve port and valve flange of the fully inflated, buoyant gastric implant of FIG. 20A.

FIG. 20C is a close-up cross-section view of the valve assembly, valve port and valve flange of the deflated, buoyant gastric implant of FIG. 20A.

### DETAILED DESCRIPTION OF THE INVENTIONS

The present invention includes gastric implants and methods for restricting the capacity of a patient's stomach and to stimulate nerves in the stomach to provide a sense of satiety and to treat obesity. As used herein, the term “gastric implant” describes a buoyant implant or implants that are configured for implantation within the stomach. Such implants are further configured to be inserted into the patient while in a first, smaller cross-sectional configuration and then expanded to a second, larger cross-sectional configuration. The implants are configured to be depressurized and collapsed to the first cross-sectional configuration for removal once their presence is no longer therapeutically beneficial.

In certain configurations, a buoyant gastric implant is implanted into the body of a patient such as a human, mammal, or other animal. The gastric implant may be disposed within the stomach. The gastric implant may be selected from one or more shapes comprising, but not limited to, a sphere, an egg, an ovoid of revolution, a rounded rectangle, a rounded triangle, a ring or inner tube, or the like. A ring shape (note that as used herein the term “ring” comprises both circular and non-circular shapes, and both open and closed configurations), an oval shape, a C-shape, a D-shape, a U-shape, an S-shape, a helical or coil shape, a cage shape, a wire stent shape and other shapes. The gastric implant can be implanted by swallowing, or by endoscopic placement with an esophageal instrument, as those of skill in the art will appreciate.

A variety of different implant locations are described below, including, but not limited to, entirely within or around the stomach. Those of skill in the art will appreciate that the present implants may be implanted anywhere within or around the stomach, the intestine, the esophagus, and the like. Multiple implants can be placed at different locations within the stomach, the esophagus, or the intestine. Further, the implants described herein can also be used in combination with other surgical procedures, such as Gastric Bypass, VBG, Duodenal Switch, etc.

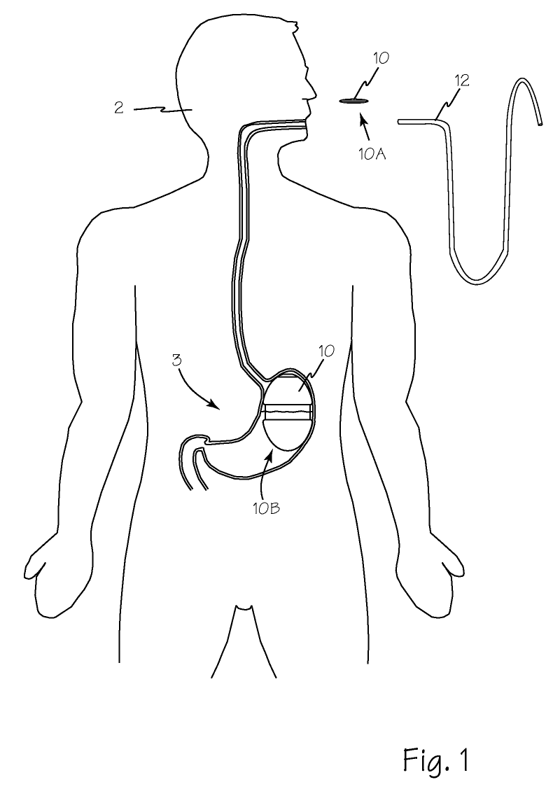

Referring now to FIG. 1, patient 2 is to receive a buoyant gastric implant to help reduce food intake and thus lose weight. Gastric implant 10 is provided in a collapsed configuration 10A ready for implant. Implant 10 is delivered to any suitable body cavity in a patient such as stomach 3 using any suitable delivery method and apparatus such as an endoscope sheath or catheter such as catheter 12. Once gastric implant is situated in the body cavity of choice, implant 10 is inflated or filled with any suitable fluid to achieve buoyant implant configuration 10B. Upon completion of a suitable period of weight loss, gastric implant 10 may be removed by locating the implant in body cavity 3, depressurizing the implant and removing the implant using any suitable technique and apparatus.

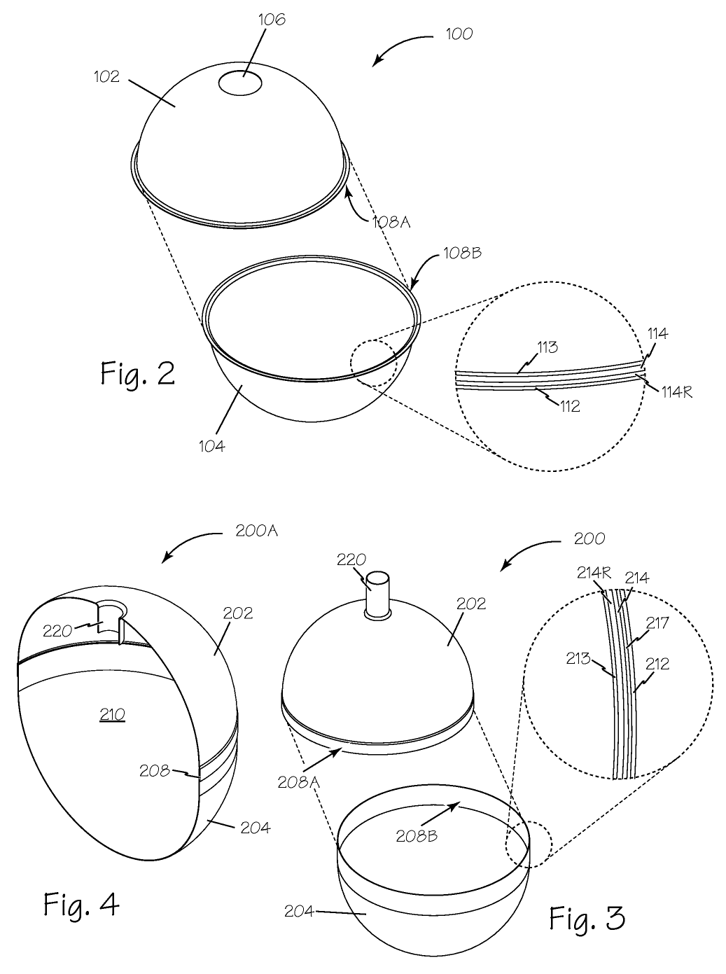

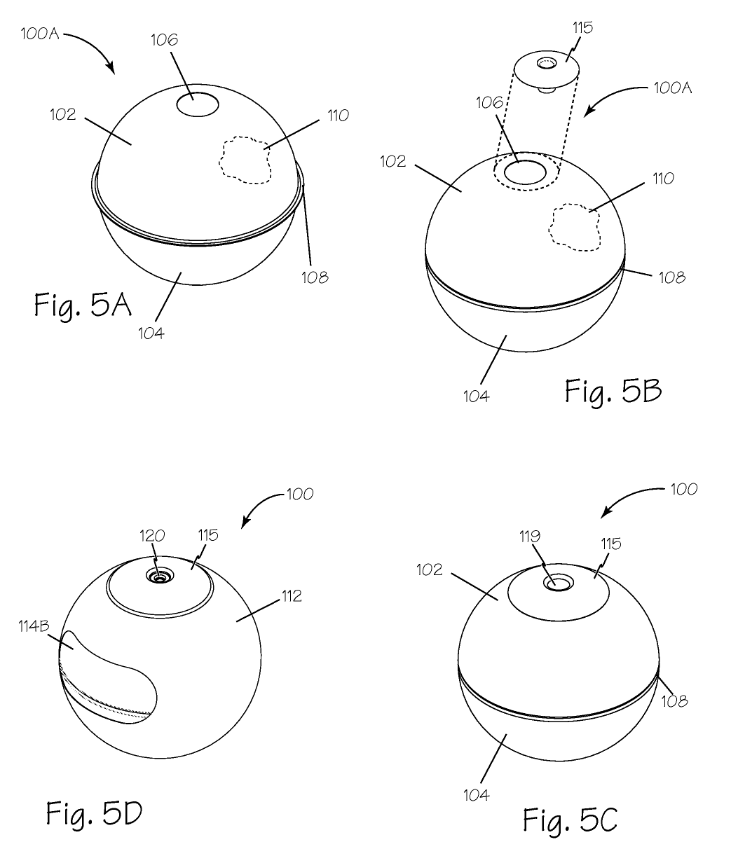

FIGS. 2 and 5A illustrate, in oblique view, a gastric implant 100 comprising a first or upper section, membrane, shell or segment 102, a second or lower section, membrane, shell or segment 104, an opening 106, an upper interface flange 108A and lower interface flange 108B.

Segments 102 and 104 of the device 100 are fabricated from one or more layers, coatings or films of biocompatible polymeric material. The thickness of the polymeric material layers can range from about 0.001 inches to about 0.250 inches with a preferred range of about 0.005 to about 0.025 inches. The first segment 102, the second segment 104, or both can be fabricated using processes such as but not limited to, injection molding, thermoforming, blow molding, liquid injection molding, stretch or extrusion blow molding, or the like. Inner layer or coating 113 is a generally gas impermeable layer, structural layer 114 may be any suitable material to provide structural integrity, and outer layer 112 is a generally lubricious and erosion resistant coating which may be used to minimize irritation of internal tissues, lubricate and protect structural layer 114.

First and second segments 102 and 104 can be welded, clamped, or bonded together at the interface region and trimmed to size. At least one segment such as upper segment 102 comprises the pre-cut opening hole 106 for engaging a valve port, such as valve port 115 of FIG. 5B, in later process. The first segment 102 and the second segment 104 can be fabricated from materials such as, but not limited to, silicone elastomer, polyurethane elastomer, polycarbonate urethane, polyester, polyethylene, Hytrel™, Pebax™, or the like. The first segment 102 and the second segment 104 are affixed to each other at the interface region 108 such that a gas-tight seal is created between the first and second segments.

In some configurations, a buoyant gastric implant can be spherical and constructed from two layers of thin film welded/bonded together to form a finished or enclosed shell such as enclosed shell 100A of FIG. 5A. Structural layer 114 of each segment can be single or multi-coextruded thin layer films, or single layer film with reinforcement. Membrane or structural layer 114 can be treated with gas barrier coating such as PVdC, Parylene, and the like; utilizing coating processes such as but not limited to, a vapor-deposition process to form inner layer or coating 113. A reinforcement layer such as layer 114R can be in the form of a fabric, mesh, weave, knit, braid, or similar structure. Materials suitable for fabricating the wall structure can include Polyurethane/PVdC/Polyurethane or Silicon/Saranex/Silicon or any other combination of biocompatible coextruded films, the ones listed herein denoting a central reinforcement material.

A hydrophilic coating 112 such as, but not limited to, hydrophilic hydrogel fabricated from Poly 2-Hydroxyethylmethacrylate pHEMA), and polyethylene glycol (PEG), and the like, can be also added or applied to the exterior of the device to reduce wall friction with the internal walls of the gastrointestinal tract, thus reducing or minimizing resistance during implantation. The uptake of water into the hydrophilic layer 112 and incorporation of the water into its structure reduces friction and can cause a certain degree of volumetric swelling of the layer, a feature that can be adjusted in configuring the hydrophilic layer. The hydrophilic layer 112 can be dried prior to implantation; consequently, in certain configurations, the hydrophilic layer 112 can be relatively thin (from about 0.0005 inches to about 0.010 inches) when dry.

The first, or upper, segment 102 can be affixed to the second, or lower, segment 104 at their interface flange or region 108 using methods such as, but not limited to, adhesive bonding, clamping, RF welding, induction welding, or the like. The method of affixing one segment to the other is dependent on many factors such as, but not limited to, material selection, degree of stiffness allowable, material thickness, and the like.

Silicone elastomers, for example, lend themselves to adhesive or solvent bonds while polyurethanes may be more amenable to radiofrequency (RF) welding techniques.

Regardless of the shape of the device, the device can be configured such that internal volume 110 has capacities ranging from about 100 mL (cc) to about 2000 mL with a preferred range of about 400 mL to about 1000 mL. In some configurations, internal volume 110 can be self-adjustable. The internal volume 110 can also be manually calibrated as desired. In other configuration, internal volumes may be different and are specified by different shapes and performance characteristics preferred.

Referring now to FIGS. 3 and 4 illustrates first, top or upper section 202 of a gastric implant 200 comprising an integral valve port 220 and an interface region 208. First or upper section 202 can be injection molded from silicone, thermoplastic urethane, or the like forming structural layer 214. Upper section 202 can be reinforced with polymeric or metallic mesh forming one or more reinforcing layers such as layer 214R to improve radial strength or other mechanical properties.

Second, lower or bottom section 204 of a gastric implant 200 may be affixed to the upper section 202 along interface region 208. Connection of upper section 202 to lower section 204 forms finished shell 200A which defines an internal volume such as internal volume 210. Buoyant gastric implant 200 can include one or more internal gas impermeable layers or coatings such as layer 213, one or more reinforcement layers 214R and one or more outer coatings or layers for structural characteristics or biocompatibility such as layers 217 and 212.

FIG. 4 illustrates, in cross-section buoyant gastric implant 200, first section 202 is affixed to the second section 204 to define internal volume 210. Upper section 202 can be affixed to the lower section 204 by adhesive bonding, welding, integral forming, clamps, mechanical fasteners, or a combination thereof. The integral valve port 220 is visible in the upper section 202 in an inverted configuration, projecting inward and not outward where it might damage the gastrointestinal lining should it come in contact therewith. Any suitable valve assembly such as valve assembly 120 of FIG. 5D may be secured within valve port 220.

FIG. 5B illustrates the gastric implant 100 of FIG. 5A after being flipped, inverted, or everted, inside out, wherein the implant 100 comprises the upper segment 102, the lower segment 104, the at least one opening 106, the interface region 108, and the internal volume 110.

After welding or bonding, the assembly can be flipped inside-out via the opening hole 106 to bring the interface region 108 to the inside of assembly. This configuration is used to ensure that there are no sharp edges or hard edge protruding from the outside surface of device 100. The opening hole 106 can be cut into the upper segment 102 following manufacturing by drilling, skiving, die cutting, or the like, or it can be integrally formed into the upper segment 102 at the time the upper segment is fabricated.

FIG. 5C illustrates, in oblique view, the gastric implant 100 comprising the upper segment 102 joined to lower segment 104 along interface region 108 to form internal volume 110, valve port 115 secured to upper segment 102 through opening 106 and the exterior lubricious coating 112.

Referring to FIGS. 5B and 5C, after the “flipping”, inversion, or eversion process, an injection molded valve port 115 is affixed to the assembly where the opening hole 106 is located. For devices with Silicone wall construction, direct bonding process can be used between the device membrane and valve port using adhesives, solvent bonding, or the like. For devices with Polyurethane membrane, an RF welding process can be performed through the bottom layer. During this process, a thin insulator can be temporarily added between layers.

The valve port 115 is optional and can be eliminated, in certain configurations, with a valve being affixed directly to the opening 106. The valve port 115 can be fabricated from rigid or flexible polymer, as well as from materials such as, but not limited to, ceramic, stainless steel, titanium, cobalt nickel alloy, nitinol, and the like. The function of the valve port 115 is to provide a seat within or to which a valve can be affixed. A central lumen of the valve port is operably connected to the internal volume 110 of the implant 100.

Referring to FIG. 5D, valve assembly 120 is affixed within valve lumen 119 of valve port 115, which is, in turn, affixed to the device assembly segment which contains a suitable opening such as opening 106. The hydrophilic coating 112 can be applied to outer surface 114B of membrane or layer 114 to reducing resistance of device during implantation.

Referring to FIG. 5D, valve assembly 120 comprises a core lumen that can open and close by action of the valve and the valve core lumen is operably connected to the internal volume 110 of the implant 100. Valve assembly 120 can comprise structures such as, but not limited to, a duckbill valve, a puncturable membrane valve, a pinhole valve, a flapper valve, a tubular valve, a plug valve, a spring-loaded valve, a cross-slit valve, a ball valve, a needle valve, or any combination, thereof. Valve assembly 120 can comprise active opening devices such as motors or actuators controlled by an operator by way of a catheter or a non-invasive energy source such as, but not limited to, high-intensity focused ultrasound (HIFU), radio-frequency generation, or the like.

After testing to ensure that the assembly 100 is leak free, all gases or fluids are removed from the device such that the device wall collapses to a minimum profile. The device's wall is then rolled up, or furled, along the longitudinal axis of the device.

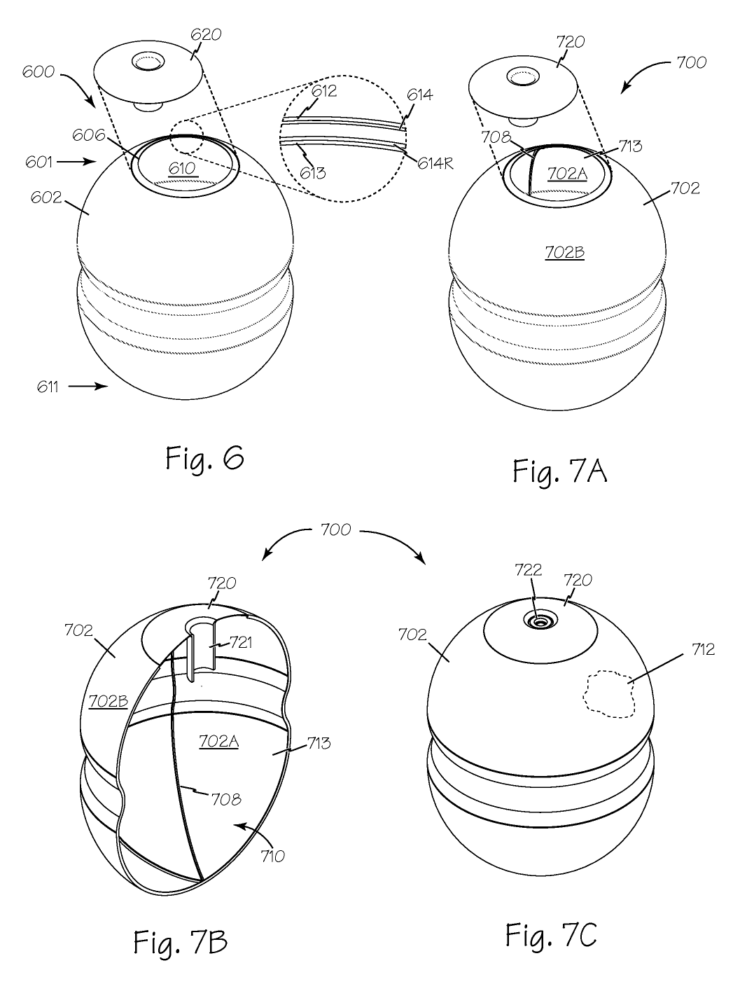

FIG. 6 illustrates, in oblique view, a gastric implant 600 comprising a single wall or unitary structure comprising a wall 602, an internal volume 610, and one or more openings 606. The one or more openings such as opening 606 may be in any suitable location, preferably in the first or second end 601 or 611 respectively.

Wall 602 is generally oval or egg-shaped in configuration and can comprise a central band that is somewhat recessed or bulging diametrically. Wall 602 may be formed of one or more layers or coatings as discussed above such as layers or coatings 613, 612, 614 and 614R. In some configurations, the wall 602 can be fabricated using dip molding or liquid injection molding with silicone elastomer, polyurethane elastomer or other materials listed as suitable for the device of FIG. 1. The opening 606 allows the molded implant 600 to be removed from the core and allows for later attachment of auxiliary components such as valves, and the like using valve ports such as valve port 620 as discussed above.

FIGS. 7A, 7B and 7C illustrate, in oblique view, a gastric implant 700 comprising a single wall or unitary structure further comprising a wall 702, an internal volume 710, one or more end openings 706, and a reinforcement structure 708.

Referring to FIG. 7A, the wall 702 can be highly elastic and have a high elongation ratio. Thus, a reinforcing structure 708 can be affixed or fabricated integrally to the wall 702. The reinforcing structure 708 can comprise polymeric or metallic ribs running parallel to the longitudinal axis of the device, running circumferentially, or both. The reinforcing structure 608 can comprise a mesh, braid, weave, knit, or other fabric structure using fibers of materials such as, but not limited to, stainless steel, PEN, PET, polyamide, polyimide, PEEK, titanium, nitinol, cobalt nickel alloy, and the like. The reinforcing structure 708 can be flexible in one or more axes. The reinforcing structure 708, in a preferred configuration is somewhat stiff and rigid in the longitudinal direction but more flexible circumferentially to permit folding and furling of the structure 708 in preparation for implantation. The reinforcing structure 708 can prohibit folding in one or more direction, it can prohibit compression or expansion in one or more direction, or it can be flexible and control only expansion while permitting flexibility and folding.

A barrier coating or layer such as layer 713 can be applied to interior surface 702A of wall 702, to exterior surface 702B of the wall 702, or both. The coating 713 can be configured to adjust the permeability of the wall 702. The wall 702 can comprise macroscopic or microscopic openings, holes, or fenestrations through which liquids, gasses, or both can flow. The coating or coatings such as layer 713 can span, or plug completely or partially, the holes or fenestrations in the wall 702 or it can work in conjunction with the wall 702 to decrease gas permeability. Permeablity controlling coatings such as layer 713 can comprise materials such as, but not limited to, expanded polytetrafluoroethylene, Parylene, or the like.

Referring to FIG. 7B, the valve port 720 is affixed to the one or more openings 706. The central lumen, valve lumen 721, of the valve port is operably connected to the internal volume 710 of the implant 700. Valve port 720 can be RF welded to the wall 702 at the opening 706.

FIG. 7C illustrates buoyant gastric implant 700 following affixation of a valve assembly 722 to the valve port 720. The implant 700 can further comprise a hydrophilic coating 712 on at least a portion of the exterior surface of the wall 702. The valve assembly 722 can be of the same type as described for the device in FIG. 5. The valve assembly 722 can be affixed to the valve port 720 using the same methods as described in FIGS. 3 and 4. Buoyant gastric implant 700 is tested for fluid and gas impermeability. It is then evacuated of fluid, gas or liquid, or both, to permit folding along the longitudinal axis and furling.

The section, membrane or shell components of the buoyant gastric implants 100, 200, 600 and 700 can be fabricated using similar techniques, including dip molding, stretch blow molding, extrusion blow molding, injection molding, liquid injection molding, thermoforming, and the like.

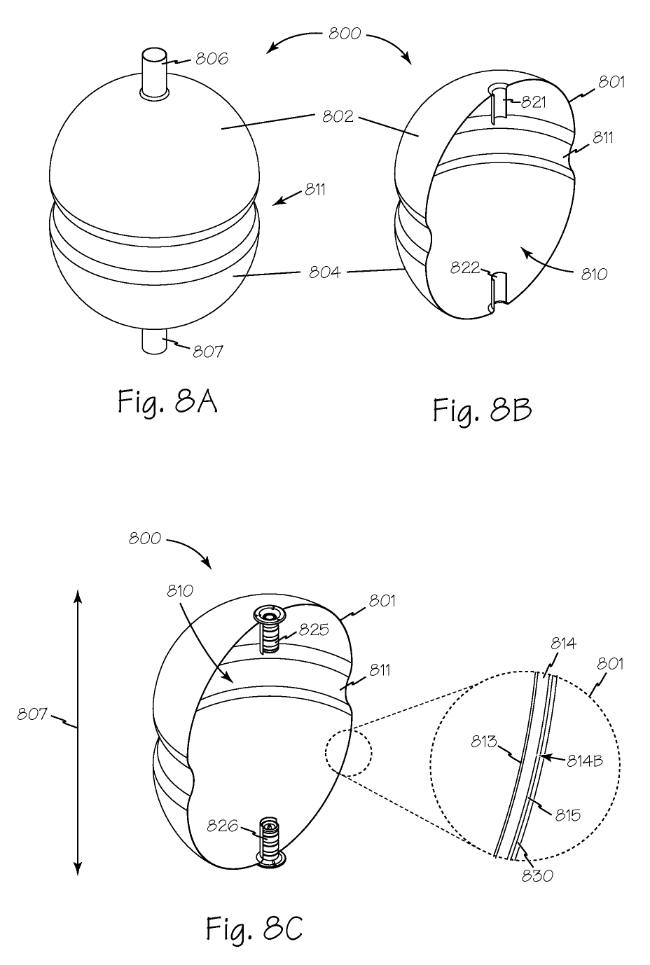

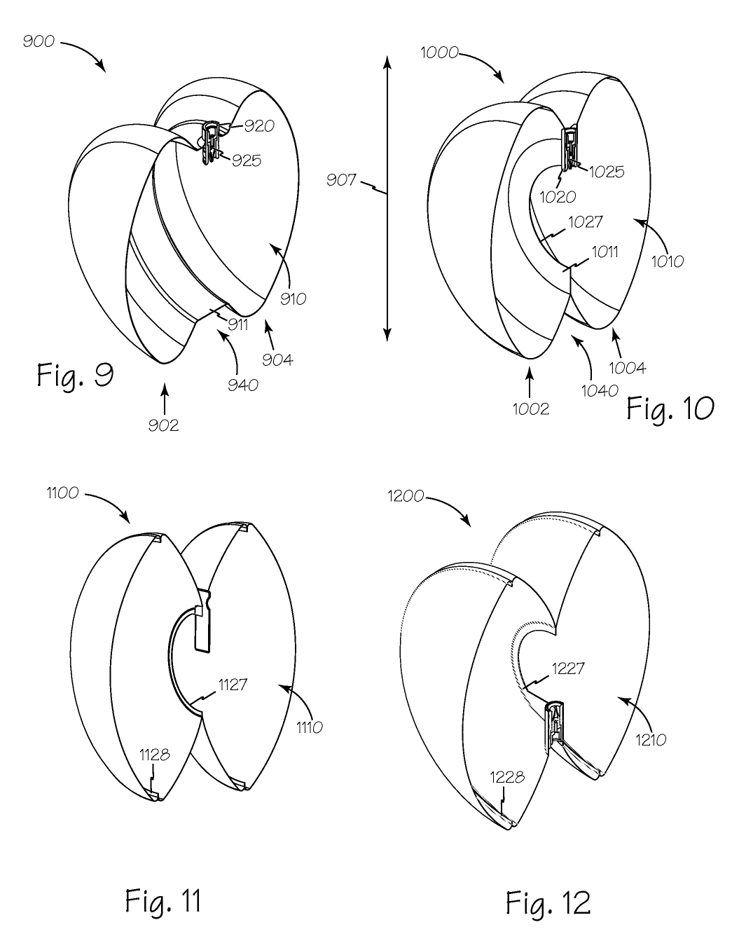

FIGS. 8A, 8B and 8C illustrate an oblique view of buoyant gastric implant 800 fabricated from thin, stretchy, blow-molded materials. Buoyant gastric implant 800 comprises a first or upper portion 802, a second or lower portion 804, a first valve port 806, a second valve port 807, a central interface region 811, and an internal volume 810. Wall 801 may have a thicknesses that can range from about 0.0005 to about 0.1 inches, or greater with a preferred range of about 0.001 to 0.005 inches.

First portion 802 and second portion 804 may be integrally formed, or they can be formed separately and affixed to each other at the central interface region 811. First valve port 806 and second valve port 807 can be formed integrally with the first portion 802 and second portion 804, respectively, or valve ports may be affixed in a secondary operation from separate components as discussed above.

First portion 802 and second portion 804 can be fabricated from polyethylene terephthalate (PET). Processes such as stretch blow-molding or extrusion blow molding can be used to achieve wall thicknesses of wall 801 in the range of about 0.001 to about 0.010 inches. After forming, valve ports 806 and 807 can be trimmed to length.

FIG. 8B illustrates a cross-sectional oblique view of the gastric implant 800 of FIG. 8A with the valve ports 806 and 807 inverted to project inwardly into internal volume 810 to form first valve lumen 821 and second valve lumen 822.

FIG. 8C illustrates a cross-sectional view of buoyant gastric implant 800 wherein a thin coating 815 has been applied to outer surface 814B of structural membrane 814. The thin layer or coating 815 can comprise materials such as, but not limited to, polyurethane, silicone, Parylene, and the like. The thin layer or coating 815 can have a thickness of about 0.00025 to about 0.010 inches with a preferred range of about 0.0005 to about 0.003 inches. As discussed above, any suitable valve assemblies such as valve assemblies 825 and 826 may be secured within first and second valve lumens 821 and 822 respectively.

An optional hydrophilic coating 830 can be applied or coated onto any suitable exterior layer such as layer 815 to reduce friction with the lining of the gastrointestinal tract. The buoyant gastric implant is evaluated or tested to confirm appropriate fluid, specifically gasses, and wall permeability of the implant 800 as well as other functional characteristics. The fluid, or gas, is next evacuated from the interior volume 810. The device 800 is then flattened, or folded, and rolled along its longitudinal axis 807. The implant 800 can be inserted into a loader to maintain the folded configuration and to facilitate introduction into the proximal end of a delivery catheter such as catheter 12 of FIG. 1.

FIG. 9 illustrates another configuration of the buoyant gastric implant 900 in oblique cross-sectional view. Gastric implant 900 can be configured to conform to the walls of the stomach or gastrointestinal tract. Buoyant gastric implant 900 can further include flutes or channels such as channel 940 running parallel to axis 907 to permit food to pass along the implant while the device occupies volume within the stomach. Buoyant gastric implant 900, as illustrated, comprises a first portion 902, a second portion 904, a central interface region 911, a valve port 920, a valve assembly 925, and enclosed volume 910. The gastric implant 900 can comprise, or be configured in, many irregular shapes such as double-lobed, double pouched, accordion shape, donut shape, flower petal shape, or the like. The accordion configuration of FIGS. 11 and 12 can facilitate easy manufacturing with a lower investment in tooling costs. This type of device further allows for a wider selection of materials.

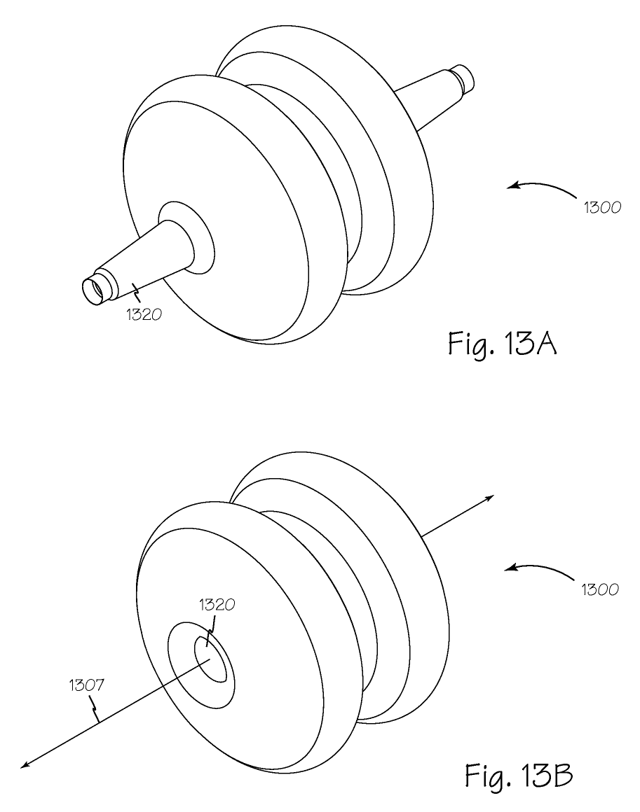

Referring now to gastric implants 900 and 1000 of FIGS. 9 and 10 respectively, the implants be constructed from two dip-molded cells affixed to each other at the interface regions 911 and 1011 by RF welding, thermal bonding, adhesive bonding, mechanical fasteners, or the like. The first portion 902 or 1002 and the second portion 904 or 1004 can be fabricated from materials such as, but not limited to, polyurethane, PEEK, polyimide, polyethylene, silicone, polypropylene, PTFE, FEP, PFA, or the like. Certain biocompatible polyurethanes include Tecothane™, Tecoflex™, Carbothane™, and the like. The first portion 902 or 1002 and the second portion 904 or 1004 can be dip-molded to construct a multi-layer wall with, for example, a polyurethane inner layer, a PVdC central layer, and a polyurethane outer layer to improve or control the gas barrier properties of the wall. Further the cell can be coated with barrier coating material such as Parylene after molding and then be inverted, everted, or turned inside out for welding and final assembly. Generally, this configuration allows for improvement in gas barrier capability of device 900 and 1000. The method further allows for incorporation of valves parallel to axis 907, of the implant to minimize the risk of the valve impinging directly on the stomach or intestinal wall or other tissue. This design further allows channels, flutes or gaps to allow food to pass through or along such as channels 940 and 1040.

In other configurations, the gastric implants such as implants 900, 1000, 1100 and 1200 can comprise four or more layers of thin film material. This construction facilitates the use of multi-layer co-extruded films such as, but not limited to, PET and EVA or PET with EVOH, and PET, for example. Such layered construction provides increased control over moisture and gas penetration. In practice, the four or more layers are welded together to form closed compartment or enclosed volume such as volumes 910, 1010, 1110 and 1210. These enclosed volumes or compartments can then be flipped inside out, or inverted, prior to final assembly at the interface region such as interface 1011. This construction prevents or minimizes exposing any sharp edges such as joint edges 1027, 1127, 1227, 1128 and 1228 on the outside of the devices 1000 and 1100 which could damage an intestinal or stomach wall or any other gastrointestinal tissue. Buoyant implants 900, 1000, 1100 and 1200 can be coated with a thin layer of polyurethane or other material for control of strength, durability, lubricity, fluid permeability, or other parameter.

FIGS. 13A and 13B illustrate a gastric implant 1300 in another configuration where a valve port such as valve port 1320 is formed integrally, or added as a secondary operation, along central axis 1307 of implant 1300.

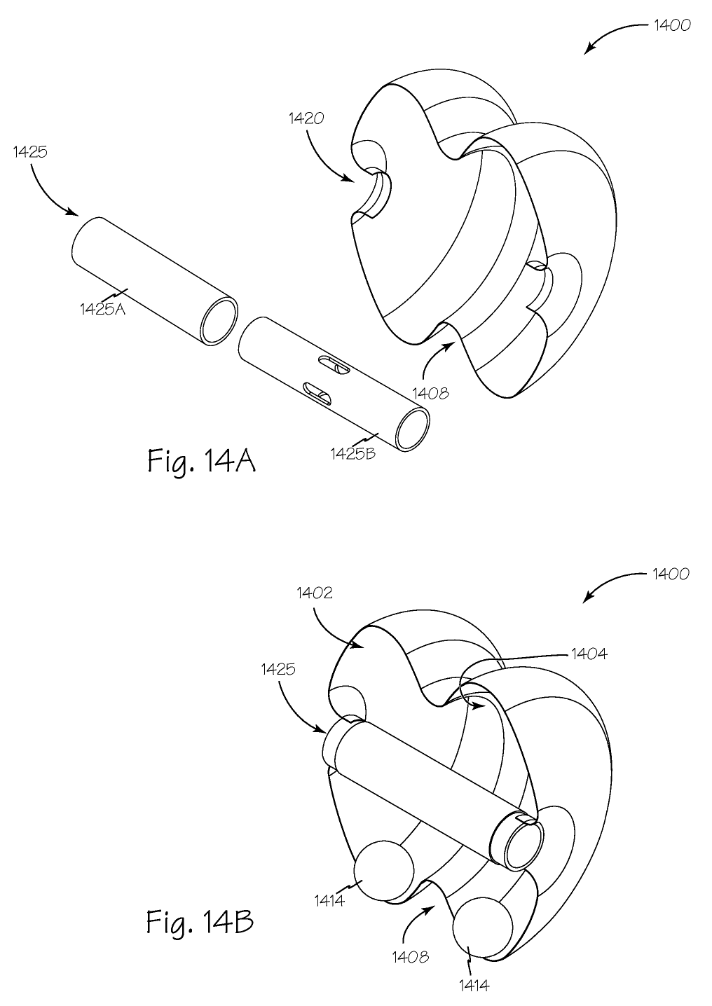

FIGS. 14A and 14B illustrates a cross-sectional oblique view of an implant 1400 having a central through lumen 1420 to engage a valving assembly 1425, and one or more orientation weights or ballast elements such as weights 1414.

Buoyant gastric implant 1400 can be weighted with ballast 1414 located in either first section 1402 or in second section 1404, so that the implant floats or rides within the patient's stomach such that the central through lumen 1420 is aligned generally along a cranial-caudal axis. This up and down orientation of the central through lumen 1420 permits solid food to pass therethrough once it is digested and forms a liquid or slurry. The through lumen 1420 can have a diameter of about 0.25 inches to a diameter of about 2 inches. The circumferential groove 1408, in this configuration, can facilitate anchoring the device in a certain position within the stomach since the stomach tissue would tend to wrap around and into the groove 1408 to some extent.

In another configuration, the gastric implant 1400 can be weighted with ballasts 1414 in both first section 1402 and in second section 1404, as illustrated, such that the implant floats or rides within the patient's stomach such that the central through lumen 1420 is aligned laterally, in the anterior-posterior direction, or in some other direction generally perpendicular to the vertical axis of the body. In this configuration, the groove 1408 formed between the first section 1402 and the second section 1402 is configured for the passage of foods following a period of temporary delay. This migration of passage of food can be aided by partial food digestion by the stomach. The circumferential groove 1408 can be provided as a single groove or the implant 1400 can be provided with a plurality of circumferential grooves 1408 with their width and depth configured for control of food passage. The number of circumferential grooves 1408 can range from about 1 to about 10. The grooves 1408 need not be entirely circumferential but are sized to permit food passage even when implant 1400 is trapped by shrunken stomach walls.

Valving assembly 1425 is composed of valve elements 1425A and 1425B and is disposed within the central through lumen 1420 and can be accessed by an instrument passed into the through lumen 1420 and pressurized to open the valve. The valving assembly 1425 can also be operated with an instrument comprising a penetrating needle or tube that projects laterally from the instrument which is inserted into the through lumen 1420.

In some configurations, vapor pressure release and gas carrier materials are utilized to enhance the volume of the buoyant gastric implant. Enhancers such as Perflourohexane, Perflouropentane or Perfouromethylbutylether are suitable oxygen carriers. The gas enhancers are configured to augment the pressure of gas or other material already present within the interior volume of the gastric implants discussed above. The gas enhancers can increase the pressure within the enclosed volume of the implant such as implants 100, 200, 300, 600, 700, 800, 900, 1000, 1100, 1200, 1300 and 1400. The gas enhancers can generate between about 1% and about 100% of the pressure within the enclosed volume of the buoyant gastric implant and preferably about 20% to about 70% of the pressure in the enclosed volume of the implants. The gas generators do not need to completely inflate the implant and partial inflation may be preferred in certain configurations, thus allowing for follow-up adjustment or breathing.

In the saturated state, Perflouropentane carries about 80% oxygen volume. These materials come in a liquid state and designed to evaporate to gas state at pre-determined temperature and vapor pressure, which can be controlled.

As any gas balloon fabricated from thin polymer film, the gastric device is subjected to gas permeation over time. When gas permeates through the coatings, membranes and layers of the implanted device, reducing the internal pressure below the vapor pressure of the enhancer(s), the enhancer will evaporate to release more gas. This process allows the internal pressure to increase up to its vapor pressure. The enhancer stops evaporating as internal pressure reaches equilibrium. This mechanism, or process, is repeated until all liquid enhancer is evaporated.

In some configurations, the buoyant gastric implant can be constructed from non-compliant or semi-compliant material(s), which allows fixed volume at or above the stomach pressure, which is approximately from 1 to 3-Psi; gauge pressure. In other configurations, a compliant material such as silicone or polyurethane can also be used for the structural layer or film. In these compliant configurations the expansion ratio of the silicone implant can be related and controlled with its internal pressure. The silicone implant can comprise thicker end walls and thinner side walls, which provide for easier insertion. When inflated up to vapor pressure, the silicone implant can be configured to enlarge to a specific size or volume. In this application, the walls are inwardly biased toward an unstressed or natural state. The shrinkage or bias of the implant back to its unstressed state works to maintain the vapor pressure at a certain level as gas leaks or migrates out of the system. As the gas leaks, the implant initially shrinks down. The internal pressure gradually drops but not instantly as in the case of non-compliant implant. However, when the gas enhancer or generator evaporates in response to the reduced pressure, the internal pressure goes back up and the implant expands. This is a form of a dynamic buoyant gastric implant, which allows fixed volume at or above the stomach pressure, which is approximately from 1 to 3-Psi; gauge pressure.

Approximately 5-20 mL of the gas generator, enhancer, or catalyst is injected into any suitable buoyant gastric implant via Luer Port from the delivery catheter. After the gas-generator or catalyst injection is completed, approximately 50 mL of ambient air would be filled the implant utilizing the same Luer Port. This ensures that all remaining gas-generator or catalyst materials inside the catheter are flushed into the implant.

Since an oxygen molecule is the small relative to other molecules, oxygen will be the first molecules to permeate through the one or more layers of the gastric implant. The gas generator molecules, for example Perflouropentane, are much greater in size than oxygen, and so are not be able to permeable through one or more of the coatings, layers or membranes of the gastric implant. As the oxygen & other gases are emptied, the gas generator will evaporate. Twenty millileters (mL) of liquid gas generator can evaporate to approximately 2,000 mL in the gas state, which is expected to keep the intragastric device inflated over the specified duration of implantation.

In another configuration, the enhancers are used as the gas-attractive elements. These gas-attractive elements can be either liquid or solid or both. These enhancers or gas-attractive elements can be de-gassed to a minimum level. The gas-attractive elements can be in the form of beads, pellets, spheres, eggs, threads or filaments, plates or layers of material, or the like. The gas-attractive elements can be solid or hollow.

As the gas attractive element is deposited into the intragastric device inside the stomach, the gas attractive element attracts other gases from the surrounding area to fill in the device.

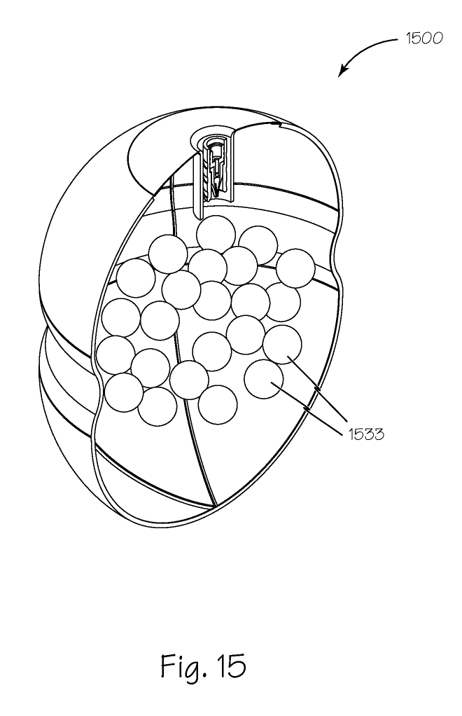

In another configuration, any suitable buoyant gastric implant such as implants 100, 200, 300, 600, 700, 800, 900, 1000, 1100, 1200, 1300, 1400 and 1500 may be filled, or partially filled, with smaller balls, balloons, pellets, or the like such as pellets 1533 in buoyant gastric implant 1500 of FIG. 15. These spherical hollow pellets, balls, balloons or other low density elements are approximately 0.375″ (range of 0.1 to 0.5 inches) diameter; constructed from biocompatible, and optionally biodegradable, polymers. These pellets are mechanically configured, and have sufficient strength, to maintain their shape against external pressure. Pellets or balloons 1533 can be filled with nothing, they can be low density solid, or they can be filled with gel, liquid, gas, or the like. The balloons or pellets 1533 can comprise only gel or solid polymer capsules.

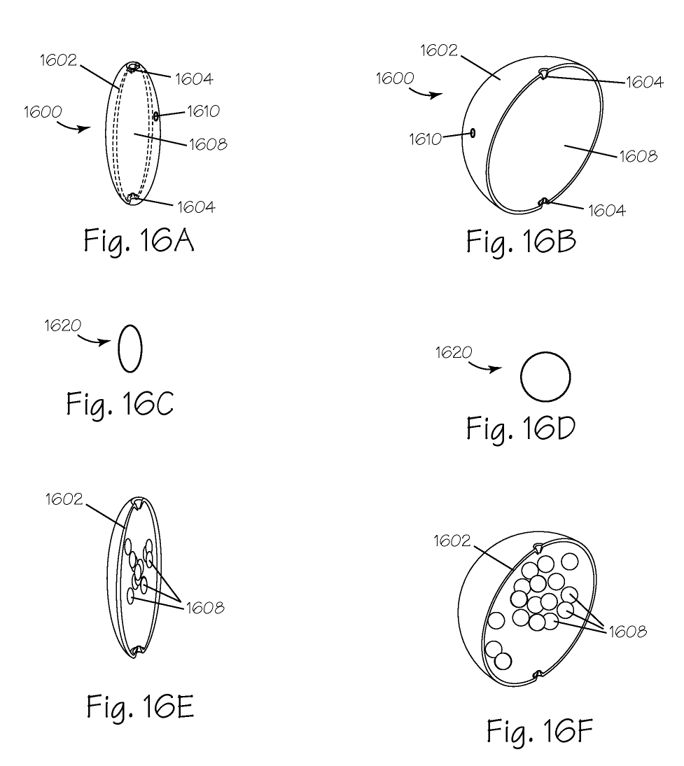

FIG. 16A is an oblique cross-sectional view of a gastric implant 1600 prior to expansion and FIG. 16B is after expansion. The implant 1600 comprises a shell 1602, one or more self-activating inlet ports 1604, and an interior volume 1608.

Referring to FIG. 16A, the polymer shell is fabricated in the elliptical shape to achieve a small insertion profile and easy implantation. The shell 1602 can be biodegradable in certain configurations and non-dissolving in other configurations. The shell 1602 may include one or more self-activating inlet ports 1604. The fluid inlet ports 1604 can range in diameter from about 0.001 inches to about 0.5 inches and are activated by the expansion state of the implant. Full expansion of the implant closes the fluid inlet port. These self-activating inlet ports such as ports 1604 can be single direction or one-way valves and configured to allow water to flow into the interior volume 1608 of the shell 1602. In the illustrated configuration of FIG. 16A, fluid flow into or out of shell 1602 is not impeded by any type of valve.

FIG. 16B is an oblique cross-sectional view of a gastric implant 1600 after expansion. The implant 1600 comprises the shell 1602, the one or more self-activating inlet ports 1604 and the interior volume 1608. The shell can further include one or more radiopaque markers such as markers 1610.

Referring to FIG. 16B, in some configurations, the shell 1602 is elastomeric and stretches. In other configurations, the shell 1602 is malleable or plastically deformable and expands but does not return to its original dimensions when internal pressure is removed. The shell 1602 wall thickness is beneficially calculated and predetermined so that as the shell 1602 is expanded, it will change from an elliptical shape to spherical shape with uniform wall thickness all around.

In some configurations, as the shell 1602 is expanded, the wall stretches from the mid-point of the shell 1602 longitude thus acting as a linkage mechanism to force the self-activating inlet ports to shut-off. By having the inlet ports shut off at a certain amount of fluid uptake, the implant 1600 becomes size limited and cannot over-expand. However, should a leak in the shell 1602 occur and the implant 1600 reduces in size, the inlet ports will re-open to allow intake of additional fluid.

The radiopaque markers such as marker 1610 can be integrated into the fluid inlet ports 1604, or fabricated into the shell 1602. The radiopaque markers can be fabricated from tantalum, gold, platinum, platinum-iridium, and the like. The radiopaque markers can also comprise barium sulfate or bismuth sulfate compounded into the material of the shell 1602.

FIG. 16C illustrates an oblique view of pellets 1620 configured for space filling within the gastric implant 1600 before uptake of water and swelling from a first, smaller size to a second, larger size. The pellets 1620 can comprise swellable hydrogel materials which increase in size with the absorption of water or other liquids. The pellets 1620 can be round, oval, cubic, pyramidal, or other suitable shape. The pellets 1620 can be coated or encapsulated to ensure that they do not stick together or to assist with governing geometry during and after expansion. In other configurations, the pellets 1620 can comprise swellable foam or other material such as a poly methyl-cellulose structure to increase size upon exposure to water or other liquid. In yet other configurations, certain foam materials can be used which are temperature-sensitive or chemically activated to increase in size.

FIG. 16D illustrates an oblique view of the pellets 1620 in their second, larger, swollen configuration. The pellets 1620 are configured to be swollen up to about 2× to 10× their original size. The pellets 1620 can expand, in certain configurations, up to about 0.1 to about 0.5 inches in major dimension. In a preferred configuration, the pellets 1620 are configured to expand from egg-shaped to circular following uptake of water or other liquid. In some configurations, the pellets can be configured with non-permeable expandable outside skins with only a portion of the skins capable of fluid or liquid permeability, such as a small region at the ends, or poles, of the pellets. In other configurations, the pellets can be in the form of threads, filaments, plates, or other structures.

FIG. 16E illustrates an oblique view of the implant 1600 of FIG. 16A, comprising a plurality of the pellets 1620, before swelling, in it's the first, smaller size configuration.

Referring to FIG. 16E, the gastric implant 1600 is substantially, dry in its interior volume 1608 and the pellets 1620 are unexpanded. Throughout this document, substantially is defined as functionally, true, not imaginary, or essentially.

FIG. 16F illustrates an oblique view of the implant 1600, filled with the pellets or beads 1620, in its second, expanded configuration. Fluid can be injected through a valve assembly 1606 allowing fluid uptake by the pellets 1620. The pellets 1620 expand to cause the shell 1602 to expand to its second, larger diameter and generally spherical configuration. In another configuration, fluid can flow into the internal volume 1608 through holes or fenestrations in the shell 1602 and allow the pellets 1602 to increase in size.

Removal of the gastric implant 1600 from a patient entails cutting open the shell to permit spillage of the hydrogel pellets 1602 into, and eventually out of, the patients digestive system, causing the implant 1600 to deflate. The deflated device 1600 can be removed from the patient through the esophagus and their mouth.

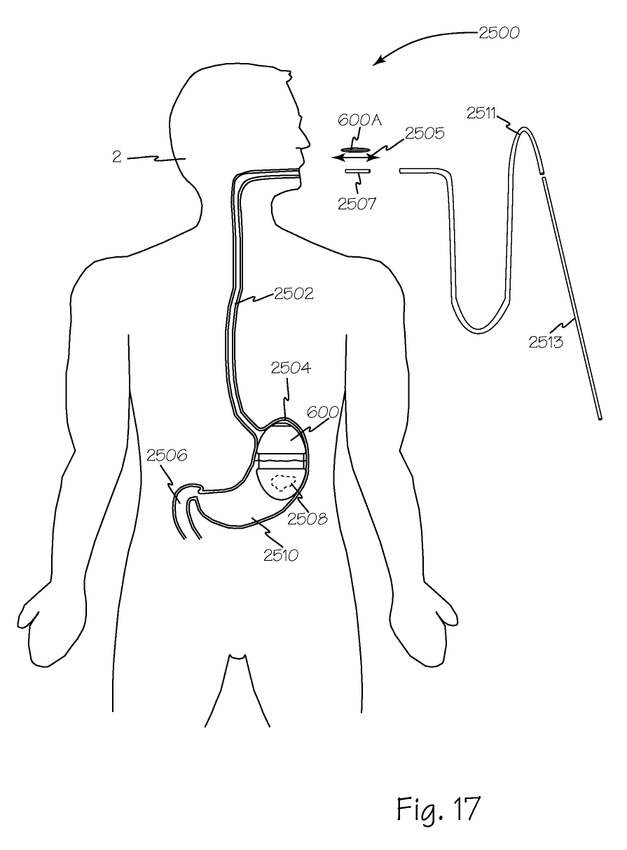

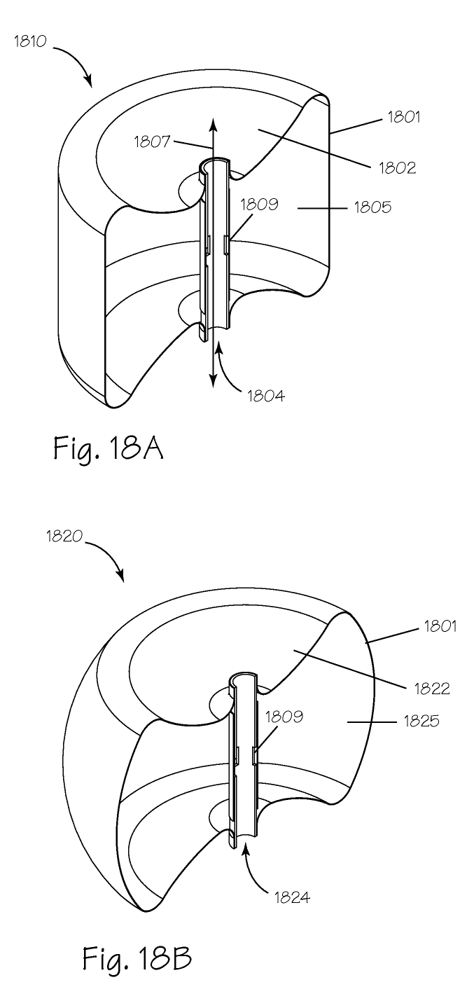

FIG. 17 illustrates a gastric implant 600 having been placed within the stomach 2504 of a patient 2500. The gastrointestinal tract of the patient 2500 further comprises the esophagus 2502 and the duodenum 2506, the latter of which leads to the lower intestinal tract. The implant 600 is buoyant and rides near the surface of any liquid or other material 2510 that represents the contents of the stomach 2504. Since the stomach walls are stretchable, the device 600 generally will migrate to the upper part of the stomach, ideally just below the esophageal sphincter where food enters the stomach. The implant 600 serves to divide off a small volume or compartment where food initially resides prior to being broken down, following which it moves past the implant 600 and into the rest of the gastrointestinal tract.

FIGS. 18A and 18B illustrate oblique cross-sectional views of buoyant gastric implant 1810 and 1820 respectively. Buoyant gastric implants 1810 and 1820 represent variations of annular construction forming food containment regions 1802 and 1822 respectively. Buoyant gastric implant 1810 and 1820 are generally cylindrical and configured to engage with the walls of the stomach to block food from passing around the outer walls 1801. Buoyant gastric implant 1810 and 1820 operate to divide the stomach into two compartments, an upper and a lower compartment. Each gastric implant includes concave upper portion food containment regions 1802 and 1822 respectively, which serve as a funnel or food containment portion. The central orifice 1804 and 1824 through which food can migrate over time, or following partial digestion by the stomach, is generally centered on the longitudinal axis of gastric implants 1810 and 1820 and food is directed into the central orifices 1804 and 1824 by the funnel shaped food containment area regions 1802 and 1822 respectively. The valves 1809 are accessed by a port, or ports, in the central orifice and can be operated by an instrument inserted into the central orifice with laterally projecting inflation or access tubes to inflate the interior volume 1805 and 1825 with gas or other buoyancy generating media (not shown). The implant of FIG. 18A can be placed within the stomach where its longitudinal axis 1807 is parallel to that of the body. Thus, the food containment regions 1802 and 1822 are oriented upward toward where the esophagus empties into the stomach and temporarily trap food in the upper stomach compartment created by the implants 1810 or 1820. Over time, the food can migrate past or through the implants through the central orifice or around the exterior walls of the implant.

The size and/or configuration of the present implants, as well as the function of the valve assembly, can be adjusted post-implantation through one of many techniques, including minimally invasive techniques and completely non-invasive techniques. For example, minimally invasive techniques include endoscopic, laparoscopic, percutaneous, etc. Completely non-invasive techniques include magnetic resonance imaging (MRI), high-intensity focused ultrasound (HIFU), inductive heating, magnetic induction, a combination of these methods, etc. The implant may be adjusted at a time to change its shape, size or valve configuration. For example, the valve can comprise meltable element that heats and dissolves upon application of electromagnetic or ultrasound energy. An antenna can be comprised by the valve to facilitate focusing energy onto the valve to perform the opening or closing. Such valve melting can facilitate opening of the valve, collapse of the system, and removal from the patient. As used herein, “post-implantation” refers to a time after implanting the implant and closing the body opening through which the implant was introduced into the patient's body.

Also as discussed above, the present implants may be implanted in any of a variety of ways, such as during a traditional open procedure, or endoscopically, or laparoscopically, or percutaneously, or through another type of procedure. First the implant is supplied in its package. The implant is preferably sterilized and delivered in a single or double aseptic package. In the illustrated configuration, the implant can be provided in its undeformed, unstressed state to maximize shelf life. In another configuration, the implant can be provided completely packaged within the delivery catheter in a ready-to-use condition. The pre-packaged inside the catheter configuration minimizes preparation of the device on the part of the implanting physician or their staff, prior to use. As delivered in undeformed configuration, the implant is deflated of any internal material, fluid, gas, etc.

In use, a gastric implant such as implant 600 of FIG. 17 is folded along longitudinal axis 2505 using a plurality of folds to pre-implant configuration 600A. Generally the number of folds is between 1 and 10 with a preferred number of 2 to 6. Once folded and furled, implant 600A can be advanced into any suitable loader, such as loader 2507 which restrains or constrains the implant inside an axially elongate structure. Implant 600A can be loaded into any suitable delivery catheter such as catheter 2511 with the aid of loader 2507. Use of a prepackaged configuration obviates the need for the user to perform the previously mentioned steps because they were performed at the factory.

Implant 600A is advanced into the mouth of patient 2 and then through the esophagus and into stomach 2504 by way of delivery catheter 2511. The implant can be placed using trans-esophageal endoscopy to aid in visualization, although fluoroscopic guidance may also be beneficial. Once implant 600 reaches the implantation site in stomach 2504, the implant is advanced out the distal end 2511D of the delivery catheter using a pusher such as pusher 2513 or other suitable mechanism such as a retractable cowling. Once located, filler 2508 such as fluid, liquid or gas, can be injected into the gastric implant by way of the delivery catheter or another catheter to fill the internal volume and generate the desired amount of buoyancy. The fluid or media can be injected through one of the valves affixed to the implant. Once the position and configuration of the device is confirmed, the delivery catheters and esophageal endoscopes can be removed from the patient.

In other configurations, the implant comprises a dried hydrogel material within its interior volume. The valve or a portion of the shell of the implant is configured to absorb liquid naturally, or passively, from the gastrointestinal tract, which is filled with water, hydrochloric acid, and food. Upon absorption of a pre-determined amount of water, the hydrogel material, which can be in the form of hollow spheres, a mass, or other structure, swells to a much larger size and fills the space of the implant with buoyant material. The amount of water absorption can be controlled by the amount of hydrogel loaded into the interior volume of the implant or by the use of a valve that can be shut off once the correct buoyancy or size has been reached.

In yet other configurations, the interior volume of the implant comprises a gas attractive element. The valve or other portion of the shell of the implant can be configured to absorb gas from the fluid or contents of the stomach. The gas permeability can be controlled by the structure of the valve or the barrier matrix of the implant walls. Gas can permeate into the interior volume of the implant in a passive manner or in a controlled way by the use of concentration gradients across a barrier membrane. Catalytic media within the interior volume of the implant can then react with the gas which has migrated in from the stomach contents and cause additional gas to be generated, raising the gas pressure within the implant to the desired, or pre-determined level. The shell can be configured to allow the gas to migrate in but not allow the gas and secondary gas generated by the catalyst to migrate outward.

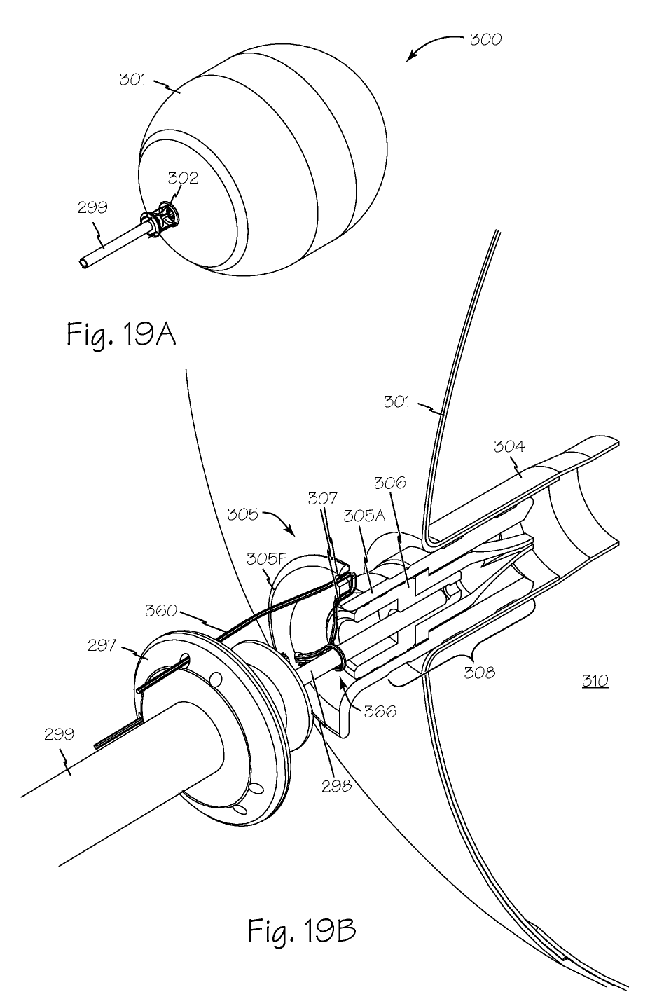

Referring now to FIGS. 19A and 19B, buoyant gastric implant 300 is illustrated full inflated with fill line 299 engaging valve 302.

FIG. 19B is a cutaway close-up view of the details of the fill line to valve connection. Fill line 299 includes a generally stiff internal lumen 298 which engages valve core 306 to convey gas into internal volume 310. Implant 300 is formed by inflatable shell 301. Shell 301 may be formed of one or more layers as discussed above and includes an integral valve port 304. Valve assembly 305 is secured within valve port 304 to seal internal volume 310. Valve assembly 305 includes valve body 305A with outer flange 305F and valve core 306. Valve body 305A is secured within valve port 304 using any suitable adhesive. To simplify removal of the gastric implant, adhesive material is only applied along attachment portion 308 of valve body 305A leaving flange 305F and the neck of the valve body free of adhesive.

During assembly of gastric implant 300, fill line 299 and valve assembly 305 are engaged using one or more sutures such as suture 360 looped around internal lumen 298, through valve flange holes 307 and then through fill flange 297. Upon insertion into a body, gas is introduced to inflate gastric implant 300. When implant 300 is fully inflated, internal lumen 298 is withdrawn out of valve core 306 and past suture loops such as loops 366 which disengages sutures 360. Sutures 360 are pulled free through valve flange 305F and fill flange 297 which disengages fill line 299 from gastric implant 300, leaving the gastric implant installed in the selected body cavity.

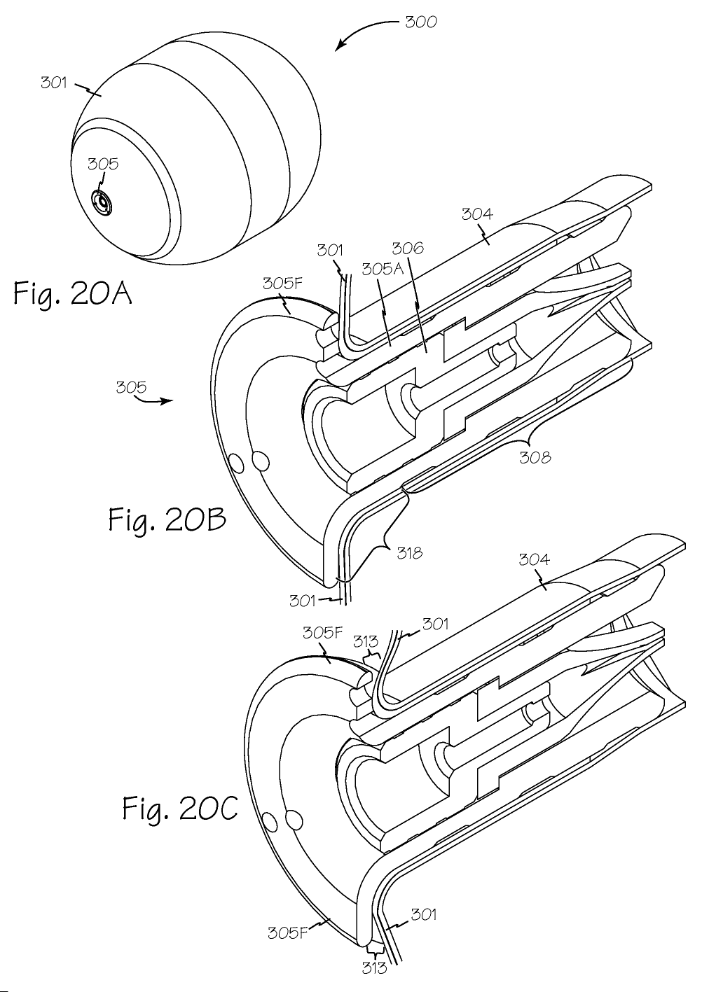

Referring now to FIGS. 20A, 20B and 20C, buoyant gastric implant 300 is illustrated with inflatable shell 301 fully inflated through valve assembly 305.

FIG. 20B illustrates the engagement between valve assembly 305 and valve port 304. During installation of valve assembly 305 into valve port 304 adhesive is only used on portion 308 of valve body 305A. This leaves portion 381 unattached to inflatable shell 301. With shell 301 fully inflated, shell 301 is in close contact with valve body portion 318 and flange 305F as shown.

To remove gastric implant 300 from a body, the implant is deflated. Upon deflation, unadhered portion 318 of the valve body separates from shell 301 forming gap 313 between flange 305F and shell 301. Gap 313 may be engaged by any suitable surgical tool such as a snare or grasping tool to remove implant 300 from a body.

While the preferred configurations of the devices and methods have been described in reference to the environment in which they were developed, they are merely illustrative of the principles of the inventions. The elements of the various configurations may be incorporated into each of the other species to obtain the benefits of those elements in combination with such other species, and the various beneficial features may be employed in configurations alone or in combination with each other. Other configurations and configurations may be devised without departing from the spirit of the inventions and the scope of the appended claims.

I claim:

1. A buoyant gastric implant comprising:
an expandable shell having an interior surface and in exterior surface enclosing an interior volume with at least one opening through the shell in fluid communication with the interior volume;
a valve port affixed to the shell through the opening in fluid communication with the interior volume;
a valve assembly affixed within the valve port in fluid communication with the interior volume;
a gas control coating on the interior surface of the shell; and
a lubricious coating affixed to the exterior surface of the shell and valve port.

2. The device of claim 1, further comprising:
a delivery catheter, wherein the delivery catheter is configured to introduce the device into the gastrointestinal tract of a patient in a first, small diameter configuration and to allow the device, once deployed in the gastrointestinal tract, to expand to a second, larger diameter configuration.

3. The device of claim 1, further comprising a loader configured to introduce the device into the delivery catheter.

4. The device of claim 1 further comprising:
gas generator component in at least a portion of the interior volume.

5. The device of claim 4 wherein the gas generator component comprises:
a solid material for generating gas when the pressure in the interior volume is low.

6. The device of claim 4 wherein the gas generator component comprises:
a liquid material for generating gas when the pressure in the interior volume is low.

7. The device of claim 6 wherein the gas generator component is selected from the group comprising:
Perfluoropentane, Perfluorohexane or Perfouromethylbutylether

8. The device of claim 1 wherein the lubricious coating comprises a hydrophilic hydrogel.

9. The device of claim 2 further comprising:
an injection port to permit gas or fluid injection into the interior volume of the shell by way of the delivery catheter.

10. The device of claim 1 further comprising:
a fluid injection port to permit injection of gas or fluid into the interior volume of the shell by way of a secondary or adjustment catheter.

11. The device of claim 1 further comprising:
a plurality of beads or pellets within the interior volume, wherein liquid introduction into the interior volume causes the beads or pellets to swell in volume, thus expanding the shell.

12. The device of claim 1 wherein the valve assembly further comprises:
a self-closing valve to limit intake of fluid into the interior volume once a predetermined configuration or dimension has been achieved.

13. The device of claim 1, wherein the shell is fabricated in a first configuration and then inverted inside-out to minimize any projections on the exterior of the device.

14. The device of claim 1, wherein the shell comprises integral valve ports.

15. The device of claim 5, wherein the hydrophilic material comprises at least one of polyethylene glycol and Poly 2-Hydroxyethylmethacrylate.

16. The device of claim 1, wherein the shell is stretch blow molded.

17. The device of claim 1, wherein the shell is extrusion blow molded.

18. The device of claim 1, wherein the shell is fabricated from the group of materials including PET, silicone, polypropylene, polyethylene, and polyurethane.

19. The device of claim 18 wherein the mass of swellable material comprises a plurality of pellets or beads.

20. A method of treating obesity in a patient comprising:
providing a gastric implant, having a longitudinal axis and an internal volume, in a sterile configuration;
removing fluid from the internal volume of the gastric implant;
folding the implant into one or more creases running parallel to its longitudinal axis;
furling the implant to achieve a first, small lateral cross-section;
inserting the implant into a delivery catheter;
routing the implant and delivery catheter through the mouth of a patient, through the esophagus and into the stomach;
deploying the implant in the stomach;
causing the implant to unfurl and expand within the stomach;
verifying the location and expanded configuration of the implant; and
removal of the delivery catheter leaving the implant within the stomach to take up volume and provide the patient recipient with a reduced appetite.

21. The method of claim 20 further comprising the step of injecting fluid into the gastric implant by way of a catheter.

22. The method of claim 20 wherein the implant expands due to expansion of a mass of hydrophilic material within the internal volume of the implant.

23. The method of claim 22 wherein the implant is elastomeric and is not folded prior to insertion into the delivery catheter but rather expands laterally due to increased pressure generated within the internal volume due to liquid migration or introduction therein.
